(19) United States
(12) Patent Application Publication
(10) Pub. No.: US 2025/0312024 A1
(43) Pub. Date: Oct. 9, 2025

(54) MEDICAL DEVICES, MEDICAL SYSTEMS, AND RELATED METHODS FOR PROVIDING TRACTION TO TISSUE

(71) Applicant: BOSTON SCIENTIFIC MEDICAL DEVICE LIMITED, Galway (IE)

(72) Inventor: Shobhit BANSAL, Gurgaon (IN)

(73) Assignee: BOSTON SCIENTIFIC MEDICAL DEVICE LIMITED, Galway (IE)

(21) Appl. No.: 19/170,423

(22) Filed: Apr. 4, 2025

Related U.S. Application Data
(60) Provisional application No. 63/575,165, filed on Apr. 5, 2024.

(51) Int. Cl. A61B 17/02; A61B 17/00

(52) U.S. Cl. CPC A61B 17/0218; A61B 2017/00269; A61B 2017/00995; A61B 2017/0287

(57) ABSTRACT

Medical devices, medical systems, and related methods for providing traction to tissue are disclosed herein. The disclosure includes a medical device. The medical device includes a plurality of rings. Each of the plurality of rings includes a rim that defines an opening. Each of the plurality of rings includes an element that extends through the opening of each of the plurality of rings from a first side of the opening to a second side of the opening. A first side of the element is coupled to a first side of the rim, and a second side of the element is coupled to a second side of the rim.

### CROSS-REFERENCE TO RELATED APPLICATION(S)

This application claims the benefit of priority to U.S. Provisional Application No. 63/575,165, filed on Apr. 5, 2024, which is incorporated by reference herein in its entirety.

### TECHNICAL FIELD

Various aspects of this disclosure generally relate to medical devices and systems for providing traction to tissue and related methods. In particular, aspects of this disclosure relate to medical devices and related methods for elastic members that apply traction to tissue to facilitate resection.

### BACKGROUND

Endoscopic procedures may involve removing a lesion from a body lumen. For example, endoscopic submucosal dissection (ESD) utilizes endoscopic tools to remove cancerous or other lesions from the gastrointestinal tract. An instrument, such as an electrosurgical knife, may be passed through a working channel of an endoscope in order to cut and remove the lesion. In some procedures, a lifting agent may be injected under the lesion prior to removing the lesion, so as to facilitate access by the electrosurgical knife or other instrument. A need exists for applying traction or tension to a lesion or other portion of a body lumen to facilitate medical procedures, such as ESD.

The medical systems, devices, and methods of this disclosure may help to rectify some of the deficiencies described above or address other aspects of the art.

### SUMMARY

Each of the aspects disclosed herein may include one or more of the features described in connection with any of the other disclosed aspects.

For example, the disclosure includes a medical device. The medical device may include a plurality of rings. Each of the plurality of rings may include a rim that defines an opening. Each of the plurality of rings may include an element that extends through the opening of each of the plurality of rings from a first side of the opening to a second side of the opening. A first side of the element may be coupled to a first side of the rim. A second side of the element may be coupled to a second side of the rim.

The medical device may include one or more of the following features. The element may include a film. The film of each of the plurality of rings may have a same size and shape as a respective opening of each of the plurality of rings. The film may be piercable by a distal tip of an end effector. The rim of each of the plurality of rings may have a first thickness. Each film of the plurality of rings may have a second thickness. The second thickness may be less than the first thickness. The film of each of the plurality of rings may cover an entirety of a respective opening of the plurality of rings.

The medical device may include one or more of the following features. The element may include one or more wires. The one or more wires may extend from a first end of a ring of the plurality of rings to a second end of the ring of the plurality of rings, opposite to the first end. Each of the one or more wires may include a first wire and a second wire. The first wire and the second wire may define a space therebetween. The space may be sized and shaped to receive an arm of an end effector. Each of the one or more wires may extend along a longitudinal axis of the medical device.

The medical device may include one or more of the following features. The element may include a mesh. For each of the plurality of rings, the mesh may include a same size and shape as a respective opening of the plurality of rings. The mesh may define a plurality of openings configured to receive an arm of an end effector.

The medical device may include one or more of the following features. Each of the plurality of rings may include a biocompatible material. The rim of the plurality of rings and the element of the plurality of rings may be elastic.

In another example, the disclosure includes a medical method. The medical method may include inserting a distal end of a first end effector through a first ring of a medical device. The first ring may include a first film spanning an opening of the first ring or a first plurality of elements spanning the opening of the first ring. Inserting the distal end of the first end effector may include piercing the film or extending the first end effector between elements of the first plurality of elements. The medical method may include coupling the first end effector to a target tissue in a body lumen. The medical method may include inserting a distal end of a second end effector through a second ring of the medical device. The second ring may include a second film spanning an opening of the second ring or a second plurality of elements spanning the opening of the second ring. Inserting the distal end of the second end effector may include piercing the second film or extending the second end effector between elements of the second plurality of elements. The medical method may further include coupling the second end effector to a wall of the body lumen.

The medical method may include one or more of the following features. The first ring may include the first film. The second ring may include a second film. Prior to piercing the first film and piercing the second film, the first film may cover an entirety of the opening of the first ring and the second film may cover an entirety of the opening of the second ring.

The first ring may include the first plurality of elements. The second ring may include the second plurality of elements. Each of the first plurality of elements and the second plurality of elements may include a plurality of wires. The plurality of wires may define one or more spaces between wires of the plurality of wires. Each of the one or more spaces may be sized and shaped to receive a portion of the first end effector or the second end effector. The plurality of wires may form a mesh.

In yet another example, the disclosure includes another medical device. The medical device may include a first ring. The first ring may include a first rim and a first opening. The medical device may include a second ring. The second ring may include a second rim and a second opening. The medical device may include a third ring. The third ring may include a third rim and a third opening. The first ring, the second ring, and the third ring may be arranged in a straight line. At least one of the first ring, the second ring, or the third ring may include a film that completely covers the respective first opening, second opening, or third opening.

### BRIEF DESCRIPTION OF THE FIGURES

The accompanying drawings, which are incorporated in and constitute a part of this specification, illustrate various aspects of this disclosure and, together with the description, serve to explain the principles of the disclosure.

FIG. 1 depicts exemplary medical system including an exemplary medical device according to some of the aspects of the disclosure.

FIG. 2 depicts a side view of the medical device of FIG. 1.

FIG. 3 depicts a side view of another exemplary medical device according to some aspects of the disclosure.

FIG. 4 depicts a side view of yet another exemplary medical device according to some aspects of the disclosure.

### DETAILED DESCRIPTION OF THE FIGURES

Particular aspects of the disclosure are described in greater detail below. The terms and definitions provided herein control, if in conflict with terms and/or definitions incorporated by reference.

The terms “proximal” and “distal” are used herein to refer to the relative positions of the components of exemplary medical devices. As used herein, “proximal” refers to a position relatively closer to the exterior of the body or closer to an operator using the medical device. In contrast, “distal” refers to a position relatively further away from the operator using the medical device, or closer to the interior of the body.

As used herein, the terms “comprises,” “comprising,” “including,” “includes,” “having,” “has,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.”

Further, relative terms such as, for example, “about,” “substantially,” “approximately,” etc., are used to indicate a possible variation of +10% in a stated numeric value or range.

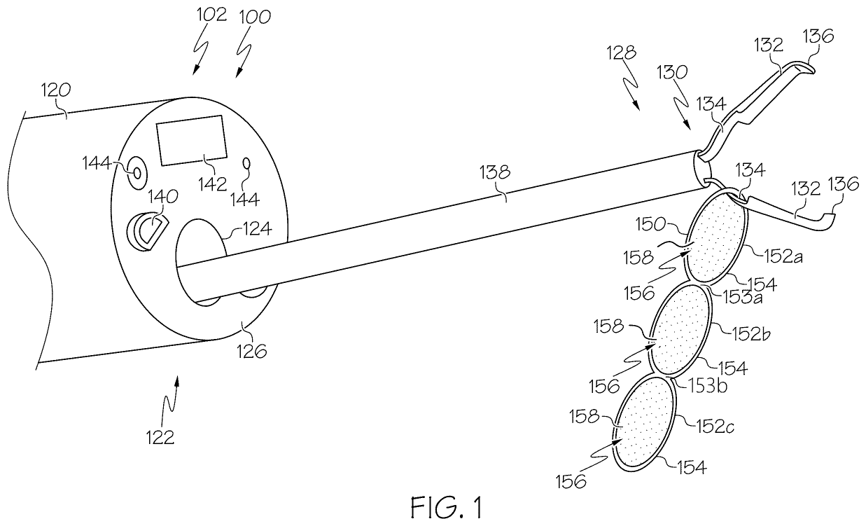

FIG. 1 depicts various aspects of an exemplary medical system 100 including an insertion device 102 and a medical device 150. Insertion device 102 may be an endoscope or any other type of scope device used for inserting into a lumen of a body of a subject. Alternatively, insertion device 102 may be a sheath or another type of device for inserting into the subject's body lumen. Insertion device 102 includes a shaft 120, and, in some aspects, insertion device 102 may include a handle (not shown). Shaft 120 includes one or more working channels 124.

The handle of insertion device 102 may be positioned at a proximal end of shaft 120. Shaft 120 may be generally tubular, and may extend from a distal end of a handle. Shaft 120 may include a distal end 122. One or more working channel 124 may extend at least from a proximal end of shaft 120 (e.g., that is adjacent to the distal end of the handle) to distal end 122. Working channel 124 may be sized and shaped to receive at least a portion of an instrument 128.

Distal end 122 may also include an illumination device or light 140 and/or include a visualization device or camera 142. Distal end 122 may further include one or more fluid channels 144. Fluid channel(s) 144 may be a fluid (e.g., air and/or water) delivery channel(s) and/or may be a suction or negative pressure channel. The handle of insertion device 102 may include one or more ports and one or more valves.

An instrument 128 may be delivered to a treatment site via insertion device 102, for example, through working channel 124. Instrument 128 may include an end effector 130 that may be actuated to transition between an open configuration and a closed configuration. For example, end effector 130 may be actuated via one or more pull wires. End effector 130 may be configured to manipulate medical device 150 and to attach to at least a portion of a target (e.g., a lesion or other portion of a wall of a body lumen) at a treatment site (e.g., a colonic wall).

End effector 130 may include a clip; however, end effector 130 is not limited to clips and may include any device or instrument configured to manipulate medical device 150 (e.g., forceps, graspers, and the like). End effector 130 may include one or more arms 132. Each of the one or more arms 132 may include notch, cinch, or space 134 configured to receive a portion of medical device 150 (e.g. such as a rim), as described below. Each of the one or more arms 132 may include a distal end/distal tip 136. Instrument 128 may also include an instrument shaft 138. A proximal end of end effector 130 may be releasably coupled to the distal end of instrument shaft 138. For example, the handle of the insertion device 102 or a handle of end effector 130 may include an actuator configured to couple and decouple end effector 130 to instrument shaft 138, as well as close (and/or open) arms 132.

FIGS. 1-4 illustrate one or more aspects of the medical device such as medical devices 150, 250, 350, as will be described in detail below. Each of medical devices 150, 250, 350 may include one or more loops or rings. Each of the one or more rings (e.g., rings 152, 252, 352) of medical devices 150, 250, 350 may include a rim (e.g., rim 154, 254, 354) defining an opening (e.g., opening 156, 256, 356). The opening(s) may contain an element/material (e.g. film 158, wires 260, and/or mesh 370) extending within the opening(s). End effector 130 may extend through and/or between the element/material. The element may extend through the opening of each of the plurality of rings, from a first side of the opening to a second side of the opening. A first side of the element may be coupled to a first side of the rim and second side of the element may be coupled to a second side of the rim.

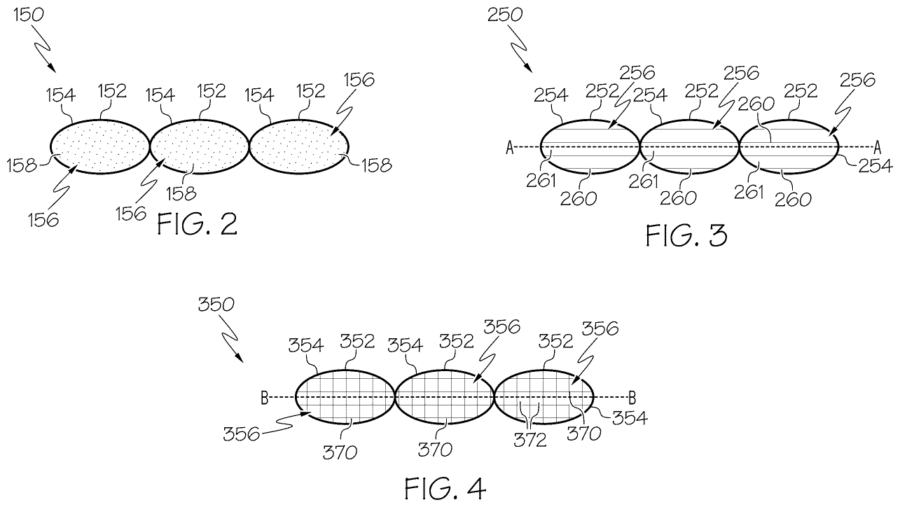

As shown in FIGS. 1 and 2, medical device 150 may include one or more loops or rings 152. For example, medical device 150 may include three rings 152. However, medical device 150 may alternatively include any suitable number of rings. One or more rings 152 may be arranged in series with one another (e.g., in a straight line, such that a straight line extends through centers of each of rings 152). Rings 152 may include a circular or elliptical shape, as shown; however rings 152 may include any shape (e.g., triangular, rectangular, irregular, trapezoidal, polygonal, etc.). Each of one or more rings 152 may include a rim 154. Each rim 154 of one or more rings 152 may intersect with or otherwise be coupled to one or more rims 154 of the other of one or more rings 152. For example, as shown in FIG. 1, a first ring 152a may intersect and/or interlock with a second, middle ring 152b and the second, middle ring may intersect and/or interlock with a third ring 152c. In other words, first ring 152a may share a first wall 153a (formed from rim 154) with second ring 152b, and second ring 152b may share a second wall 153b (formed from rim 154) with third ring 152c. Each of one or more rings 152 may define an opening 156. Each of rings 152a, 152b, and 152c may include an element (for example, a film 158) spanning an opening (for example, opening 156) of the rings 152a, 152b, and 152.

Each rim 154 may be formed from a biocompatible material, such as but not limited to, Linear Low-Density Polyethylene. Forming rim(s) 154 from Linear Low-Density Polyethylene may provide rim(s) 154 with sufficient elasticity so during an exemplary medical produce, medical device 150 may be stretched to a length approximate to or larger than a major dimension or minor dimension of a target (e.g., a lesion).

Each of the one or more rings 152 may include a film 158 (e.g., an element). Film 158 may fill, completely cover, span, and/or be positioned within opening 156. In some embodiments, film 158 may incompletely cover opening 156. For example, film 158 may cover an entirety of each opening 156. In other words, an entire inner perimeter of each rim 154 may have an edge of film 158 coupled to it. Film 158 may be the size and shape of opening 156. Film 158 may be a thin film and may include a thickness less than a thickness/height of ring(s) 152.

The thickness of film 158 may sufficiently thin to be piercable by distal tip 136 of arm 132. Alternatively, or additionally, an appropriately sized hole for arm 132 of end effector 130 may be cut through film 158 with another end effector (not shown, such as a pair of scissors or a knife) or may be pre-formed in film 158. Film 158 may be formed from a biocompatible material, such as but not limited to Linear Low-Density Polyethylene. Linear Low-Density Polyethylene may provide film 158 with sufficient elasticity to bend and contort during an exemplary procedure (for example, ESD) without experiencing wear and tear but not so much elasticity that piercing the thickness of film 158 with distal tip 136 becomes impracticable, unsafe, or possess a risk of tearing a corresponding rim 154. Film 158 may be stretchable/elastic and/or flexible, so that it may stretch along with rim 154.

Films 158 may provide additional resistance/interference between end effector 130 and rims 154. As compared with a medical device lacking film(s) 158, film 158 may inhibit rim 154 from being crushed or otherwise compressed by end effector 130, thereby inhibiting reduction in strength and/or reduction in elongation capacity of rim 154. Furthermore, film 158 may inhibit breakage of rim 154. Furthermore, because an inner diameter of each ring 152 may be substantially larger than protrusions/flanges distal tip 136 of end effector 130, film 158 may inhibit medical device 150 from slipping off of end effector 130 (e.g., off of one or more of distal tips 136). In some examples, distal tip 136 may contact and/or otherwise interact with film 158 to help to secure medical device 150 to end effector 130.

FIG. 3 depicts an alternative embodiment of medical device 150, a medical device 250. Medical device 250 may be utilized in conjunction with insertion device 102 and/or medical system 100 in the same manner as medical device 150. Medical device 250 may include any of the features of medical device 150 and may differ from medical device 150 as described below. Components and elements of medical device 250 analogous or identical to components and elements of medical device 150 share the same element number but with an additional 100 added to the element number of medical device 250 (for example, a rim 254 of medical device 250 corresponds to rim 154 of medical device 150).

In replacement of, or in addition to, a film (e.g., film 158), each of one or more rings 252 may include one or more wires 260 (e.g., an element). Although the term “wire” is used herein, it will be appreciated that “wires” 260 may be threads, filaments, strands, etc. One or more wires 260 may be positioned within an opening 256 defined by a rim 254 of each respective ring 252. One or more wires 260 may extend from a first side of rim 254 of each ring 252 to a second side of rim 254 of each ring 252 opposite to the first side. For example, as shown in FIG. 3, each of one or more wires 260 may extend from one longitudinal side to another longitudinal side of ring 252 (e.g., along a longitudinal axis of the medical device 250). For example, where a ring 252 has an elliptical shape, each wire 260 may extend approximately parallel or coaxially with a major axis of ring 252. For each ring 252, wires 260 may be symmetrical about a central longitudinal axis A of medical device 250 (e.g., symmetrical about a major axis or diameter of each ring 252). As shown in FIG. 3, no wire 260 may be coaxial with central longitudinal axis A. In alternative, one wire 260 of each ring 252 may be coaxial with central longitudinal axis A.

For example, as shown in FIG. 3, each of one or more wires 260 may extend from one lateral side to another lateral side of ring 252 (i.e., approximately perpendicularly to central longitudinal axis A). In other alternatives, wires 260 may extend at a non-zero and non-90 degree angle to central longitudinal axis A (i.e., diagonally to central longitudinal axis A). As shown in FIG. 3, each of rings 252 may have a same pattern of wires 260. Alternatively, rings 252 may have different patterns of wires 260.

One or more wires 260 may be formed from a biocompatible material, such as but not limited to Linear Low-Density Polyethylene. Wires 260 may be stretchable or flexible, along with rims 254. One or more wires 260 may define one or more spaces 261 between each of the one or more wires 260. The one or more spaces 261 may be sized and shaped to receive at least a portion of one or more arms 132. A portion of one or more wires 260 and/or rim 254 may be positioned within notch 134. In some embodiments, where distal tip 136 or one or more arms 132 is enlarged, distal tip 136 may cause one or more wires 260 to tear resulting in a larger hole to accommodate the enlarged portion of end effector 130 without destroying or substantially damaging the integrity and/or structure of remaining wires 260. Alternatively, or additionally, an appropriately sized hole for arm 132 of end effector 130 may be cut via slicing one or more adjacent wires 260 with another end effector (not shown, such as a pair of scissors or a knife).

Medical device 250 may have any of the benefits discussed above with respect to medical device 150. Wires 260 may provide additional resistance between end effector 130 and rims 254. As compared with a medical device lacking wires 260, wires 260 may inhibit rim 254 from being crushed or otherwise compressed by end effector 130, thereby inhibiting reduction in strength and/or reduction in elongation capacity of rim 254. Furthermore, wires 260 may inhibit breakage of rim 254. Furthermore, because an inner diameter of each ring 252 may be substantially larger than protrusions/flanges distal tip 136 of end effector 130, wires 260 may inhibit medical device 250 from slipping off of end effector 130 (e.g., off of one or more of distal tips 136). In some examples, distal tip 136 may contact and/or otherwise interact with wires 258 to help to secure medical device 250 to end effector 130.

FIG. 4 depicts another alternative embodiment of medical device 150, a medical device 350. Medical device 350 may be utilized in conjunction with insertion device 102 and/or medical system 100 in the same manner as medical device 150 and medical device 250. Medical device 350 may include any of the features of medical/or device 150 and medical device 250 and may differ from medical device 150 and medical device 250 as described below. Components and elements of medical device 350 analogous or identical to components and elements of medical device 150 share the same element number but with an additional 200 added to the element number of medical device 350 (for example, a rim 354 of medical device 350 corresponds to rim 154 of medical device 150).

In replacement of, or in addition to, a film (e.g., film 158), each of one or more rings 352 may include a mesh 370 (e.g., an element). Mesh 370 may be positioned within an opening 356 defined by a rim 354 of a ring 352. Mesh 370 may cover and/or fill opening 356. Mesh 370 may include a same size and shape as a respective opening 356 of the one or more rings 352. Mesh 370 may be formed from a biocompatible material, such as but not limited to Linear Low Density Polyethylene. Mesh 370 may be formed from a plurality of wires (e.g., such as wires 260 and having any of the properties of wires 260) arranged in a criss-cross pattern and/or a grid-pattern. As shown in FIG. 4, mesh 370 may include a plurality of wires that extend approximately perpendicularly to a central longitudinal axis B of medical device 350 and a plurality of wires that extend approximately parallel to central longitudinal axis B. Alternatively, mesh 370 may include wires extending at any other suitable angle (e.g., diagonally to one another). In further alternatives, mesh 370 may not be formed from wires and may instead be formed from a perforated material.

Mesh 370 may include a plurality of openings 372 (e.g., between adjacent wires or other adjacent pieces of material). Each of the plurality of openings 372 may be sized and shaped to receive at least a portion of one or more arms 132. A portion of mesh 370 and/or rim 354 may be positioned within notch 134. In some embodiments, where distal tip 136 or one or more arms 132 is enlarged, distal tip 136 may cause one or more portions of mesh 370 to tear resulting in a larger hole to accommodate the enlarged portion of end effector 130 without destroying or substantially damaging the integrity and/or structure of remaining mesh 370. Alternatively, or additionally, an appropriately sized hole for arm 132 of end effector 130 may be cut from mesh 370 with another end effector (not shown, such as a pair of scissors or a knife).

Medical device 350 may have any of the benefits discussed above with respect to medical devices 150, 250. Mesh 370 may provide additional resistance between end effector 130 and rims 354. As compared with a medical device lacking mesh 370, mesh 370 may inhibit rim 354 from being crushed or otherwise compressed by end effector 130, thereby inhibiting reduction in strength and/or reduction in elongation capacity of rim 354. Furthermore, mesh 370 may inhibit breakage of rim 354. Furthermore, because an inner diameter of each ring 352 may be substantially larger than protrusions/flanges distal tip 136 of end effector 130, mesh 370 may inhibit medical device 350 from slipping off of end effector 130 (e.g., off of one or more of distal tips 136). In some examples, distal tip 136 may contact and/or otherwise interact with mesh 370 to help to secure medical device 350 to end effector 130.

As described above, FIGS. 1-4 illustrate one or more aspects of the medical device such as medical devices 150, 250, 350. The medical devices150, 250, 350 may have a major axis and a minor axis. The major axis may be parallel with the longitudinal axis (e.g., axis A, axis B) of the medical device 150, 250, 350 and the minor axis may be perpendicular to the longitudinal axis of the medical device. The major axis may have a length of about 18.87 mm+/−0.5 mm (e.g., a total length of all of the rings 152, 252, 352 of a given medical device 150, 250, 350). The minor axis may have a length of about 4.41 mm+/−0.3 mm.

Each of the one or more rings (e.g., rings 152, 252, 352) may also have a major axis and a minor axis (e.g., rings 152, 252, 352 may have an elliptical shape). The major axis of the ring(s) 152, 252, 352 may have a length of about 6.29 mm+/−0.2 mm. The minor axis of the ring(s) 152, 252, 352 may include a length of about 4.41 mm+/−0.3 mm.

The rim (e.g., rim 154, 254, 354) of each of the one or more rings 152, 252, 352 may have a cross section with a circular or elliptical shape and may have a diameter/width of about 0.33 mm+/−0.05 mm. The interlock/intersection between adjacent rings 152, 252, 352 may have a length, parallel with a minor axis of the medical device, of about 2.0 mm+/−0.2 mm. The interlock may include a first end and a second end, positioned opposite one another. The interlock may include a first fillet at the first end and a second fillet at the second end. The first fillet and second fillet may include a diameter of about 0.78 mm+/−0.03 mm.

The opening (e.g., opening 156, 256, 356) of the rim may include a major axis and a minor axis. The major axis may include a length of about 5.85 mm+/−0.3 mm. The minor axis may include a length of about 3.75 mm+/−0.2 mm.

Features of medical devices 150, 250, 350 may be combined in any suitable manner. For example, a medical device may include rings having different properties (e.g., one or more ring(s) may have properties of one of rings 152, 252, 352, and other ring(s) may have properties of other(s) of rings 152, 252, 352). In other examples, a given ring may have properties of multiple rings (e.g., film 158 and/or wire(s) 260 and/or mesh 370).

Each of medical devices 150, 250, 350 may be elastic (e.g., stretchable) and may be manipulated by one or more end effectors (e.g., end effector 130) to be stretched along a longitudinal and/or lateral axis of medical device 150, 250, 350. For example, material(s) of medical devices 150, 250, 350 may be elastic. Film 158, wires 260, and mesh 370 may include a sufficient coefficient of elasticity to be stretched and/or pulled in tension with one or more rings 152, 252, 352 without tearing and/or breaking due to stretching.

Medical device 150, 250, or 350 may be used in an exemplary procedure (for example, ESD). As an initial step, distal end 122 of shaft 120 may be navigated within a bodily lumen so that a distalmost face 126 of distal end 122 is facing and/or adjacent to a target (e.g. a lesion). After navigating distal end 122 of shaft 120, a first instrument 128 may be extended distally through working channel 124 toward the target. After or before extending the first instrument 128, distal tip 136 of one of arms 132 of the first instrument 128 may be inserted through an opening (e.g., opening 156, 256, 356) one of one or more rings (e.g., one or more rings 152, 252, and 352). For example, inserting distal tip 136 through one of one or more rings 152 may pierce film 158 (e.g. film 158 of first ring 152a). As another example, inserting distal tip 136 through one of one or more rings 252 may cause distal tip 136 to be inserted in a space between two of the one or more wires 260. An yet another example, inserting distal tip 136 though one of one or more rings 352 may cause distal tip 136 to be inserted through one opening of plurality of openings 372. It should be understood that an operator may insert distal tip 136 through a leftmost or rightmost ring 152, 252, or 352 of the one or more rings 152, 252, or 352 so that the remaining rings are easily accessible. After inserting distal tip 136 through one of the one or more rings 152, 252, or 352, a rim (e.g., rim 154, 254, or 354) may be positioned within notch 134 to help prevent the ring 152, 252, or 352 from inadvertently disconnecting or decoupling from end effector 130.

After inserting distal tip 136 through one of the one or more rings 152, 252, or 352, an operator may couple end effector (e.g., a first end effector) 130 to a target tissue or to another surface of a wall defining a body lumen. The end effector may be coupled to the target or other tissue by actuating the end effector to transition from an open configuration to a closed configuration, clamping onto tissue of the target in the closed configuration. While coupled to one side of the target, the rim may be sandwiched or positioned between arms 132 to prevent the medical device 150, 250, or 350 from decoupling from the first end effector 130. After coupling end effector 130 to one side of the target, the first end effector 130 may be disconnected or decoupled from instrument shaft 138.

After the first end effector 130 has been decoupled from instrument shaft 138, the operator may extend a second instrument 128 (including a second end effector 130) through working channel 124 or may couple a second end effector 130 to the shaft 138 of the first instrument 128. After providing the second end effector 130 or extending the second instrument 128, the operator may insert distal tip 136 of the second end effector 130 through a different one of the one or more rings 152, 252, 352. (e.g., a ring 152, 252, or 352 on an opposite side from the ring 152, 252, or 352 previously coupled in the previous steps). After inserting distal tip 136 through the ring 152, 252, or 352, the second end effector 130 may be coupled to the target or may another portion of the bodily lumen. For example, the second end effector 130 may be coupled to a portion of the bodily lumen circumferentially opposite to the target, while the first end effector 130 may be coupled to the target. As the second end effector 130 is being coupled, the medical device 150, 250, or 350 may be pulled in tension. After second end effector 130 has been coupled, second end effector 130 may be decoupled from the corresponding instrument shaft. After the second end effector 130 has been coupled to the tissue, the tension of the medical device 150, 250, or 350 may lift a portion of the target away from adjacent walls of the body lumen to help the operator in removing the target with another medical instrument (such as scissors, electrocautery knives, ablation devices, etc.). Furthermore, during resection of the target, the medical device 150, 250, or 350 may continue to apply traction to the target, creating a tissue flap and/or aiding in removal of the target.

While principles of this disclosure are described herein with reference to illustrative examples for particular applications, it should be understood that the disclosure is not limited thereto. Those having ordinary skill in the art and access to the teachings provided herein will recognize additional modifications, applications, embodiments, and substitution of equivalents all fall within the scope of the features described herein. Accordingly, the claimed features are not to be considered as limited by the foregoing description.

What is claimed is:

1. A medical device comprising:
a plurality of rings, each of the plurality of rings including a rim that defines an opening;
wherein each of the plurality of rings includes an element that extends through the opening of each of the plurality of rings, from a first side of the opening to a second side of the opening, wherein a first side of the element is coupled to a first side of the rim, and wherein a second side of the element is coupled to a second side of the rim.

2. The medical device of claim 1, wherein the element includes a film.

3. The medical device of claim 2, wherein the film of each of the plurality of rings has a same size and shape as a respective opening of each of the plurality of rings.

4. The medical device of claim 2, wherein the film is piercable by a distal tip of an end effector.

5. The medical device of claim 2, wherein the rim of each of the plurality of rings has a first thickness and each film of the plurality of rings has a second thickness; wherein the second thickness is less than the first thickness.

6. The medical device of claim 2, wherein the film of each of the plurality of rings covers an entirety of a respective opening of the plurality of rings.

7. The medical device of claim 1, wherein the element includes one or more wires.

8. The medical device of claim 7, wherein the one or more wires extend from a first end of a ring of the plurality of rings to a second end of the ring of the plurality of rings, opposite to the first end.

9. The medical device of claim 7, wherein each of the one or more wires includes a first wire and a second wire; wherein the first wire and the second wire define a space therebetween.

10. The medical device of claim 9, wherein the space is sized and shaped to receive an arm of an end effector.

11. The medical device of claim 7, wherein each of the one or more wires extends along a longitudinal axis of the medical device.

12. The medical device of claim 1, wherein the element includes a mesh.

13. The medical device of claim 12, wherein for each of the plurality of rings, the mesh includes a same size and shape as a respective opening of the plurality of rings.

14. The medical device of claim 12, wherein the mesh defines a plurality of openings configured to receive an arm of an end effector.

15. The medical device of claim 1, wherein each of the plurality of rings includes a biocompatible material, and wherein the rim of the plurality of rings and the element of the plurality of rings are elastic.

16. A medical method comprising:
inserting a distal end of a first end effector through a first ring of a medical device, wherein the first ring includes a first film spanning an opening of the first ring or a first plurality of elements spanning the opening of the first ring, and wherein inserting the distal end of the first end effector includes piercing the first film or extending the first end effector between elements of the first plurality of elements;
coupling the first end effector to a target tissue in a body lumen;
inserting a distal end of a second end effector through a second ring of the medical device, wherein the second ring includes a second film spanning an opening of the second ring or a second plurality of elements spanning the opening of the second ring, and wherein inserting the distal end of the second end effector includes piercing the second film or extending the second end effector between elements of the second plurality of elements; and
coupling the second end effector to a wall of the body lumen.

17. The method of claim 16, wherein the first ring includes the first film and the second ring includes the second film, wherein, prior to piercing the first film and piercing the second film, the first film covers an entirety of the opening of the first ring and the second film covers an entirety of the opening of the second ring.

18. The method of claim 16, wherein the first ring includes the first plurality of elements and the second ring includes the second plurality of elements, wherein each of the first plurality of elements and the second plurality of elements includes a plurality of wires, wherein the plurality of wires defines one or more spaces between wires of the plurality of wires, and wherein each of the one or more spaces are sized and shaped to receive a portion of the first end effector or the second end effector.

19. The method of claim 18, wherein the plurality of wires forms a mesh.

20. A medical device comprising:
a first ring including first rim and a first opening,
a second ring including a second rim and a second opening; and
a third ring including a third rim and a third opening;
wherein, the first ring, the second ring, and the third ring are arranged in a straight line; and
wherein at least one of the first ring, the second ring, or the third ring includes a film that completely covers the respective first opening, second opening, or third opening.
